(19) United States
(12) Patent Application Publication
(10) Pub. No.: US 2011/0106057 A1
(43) Pub. Date: May 5, 2011

(54) CATHETER GUIDEWIRE ADVANCEMENT DEVICE

(76) Inventor: M. Samy Ahmed Hamboly, Ramadan City (EG)

(21) Appl. No.: 12/610,723

(22) Filed: Nov. 2, 2009

(51) Int. Cl. A61M 25/09

(52) U.S. Cl. 604/528

(57) ABSTRACT

A catheter guidewire advancement device comprised of an at least partially transparent housing having scale markings, a guidewire, and a thumb roller, said guidewire being coiled within said housing, said markings being aligned with the coiled guidewire such that upon extension of the guidewire, the marking adjacent to the distal end of the wire correlates with the length of wire extended from the housing.

### BACKGROUND OF THE INVENTION

1. Field of the Invention

The present invention relates generally to catheter guidewires and specifically to a catheter guidewire advancement device that may be used in cardiac and endovascular procedures and surgeries.

2. Description of the Prior Art

Medical guidewires are used in numerous medical procedures, including cardiac catheterization, interventional radiology, and endovascular surgery. Surgical guidewire may be passed through arteries to, for example, guide and place arterial catheters. Such guidewires are generally inserted through a cannula within a blood vessel and advanced to a predetermined point within the vascular system. One common type of guidewire is known as a “J type” guidewire which has a flexible “J” shaped tip which allows the guidewire to be more easily maneuvered through the vessel.

Some procedures require the use of long guidewires. In some cases, up to 56 cm may be advanced through a patient's blood vessels. Naturally, the longer the guidewire, the more susceptible it may be to contamination, especially during insertion. Long guidewires frequently become entangled during insertion. Manually untangling a guidewire may result in destroying its sterility. Moreover, because of the great length inserted, it becomes difficult for the health care provider to know how much of the wire has been extended or the approximate location of the guidewire tip in the vessel.

To solve the problems encountered in using guidewires, the present invention provides a calibrated housing for use with a coiled length of guidewire. The housing allows the user to accurately advance the guidewire a desired length into the patient.

There are other guidewire advancement devices known in the art. For example, U.S. Pat. No. 4,903,826 issued to Pearce discloses a surgical wire dispenser having a spirally formed guidewire channel. U.S. Patent Publication, Heh 20040082880 discloses a guidewire dispenser with a roller wheel. However, the prior art guidewire advancement devices fail to provide an accurate way for a physician to readily determine the amount of guidewire extended. It is also known in the art to mark the guidewire itself. However, such markings are difficult to see, interpret, or follow.

What is needed is a guidewire advancement device that is compact, easily sanitized, easily used and which readily permits a physician to accurately determine the length of guidewire extended.

### SUMMARY OF THE INVENTION

It is an object of the invention to provide a guidewire advancement device that is compact, easily sanitized, and permits a physician to accurately determine the length of guidewire extended.

The guidewire advancement device of the present invention is comprised of a partially transparent housing, a spiral passage for a guidewire, and thumb roller. In the preferred embodiment, the housing is comprised of a lower housing and an upper housing. The lower and upper housing are each further comprised of a thumb wheel opening and a tapered portion. The passage is structured and arranged such that a guidewire comprised of a proximal end and a distal end may be coiled within the housing. The thumb roller is rotatably positioned distal to the tapered portion between the upper and lower housing. When placed within the housing, the guidewire is coiled such that the proximal end extends from an end opening in the tapered portion. A portion of the guidewire may be frictionally coupled to the thumb roller such that when the roller is turned the guidewire moves.

The housing is ring shaped and has markings calibrated to the length of the guidewire extended from the housing. In the preferred embodiment, a physician using the guidewire advancement device may determine the amount of guidewire extended by comparing the position of the guidewire's distal end with a calibration marking on the housing that corresponds with the length of guidewire extended. In a preferred embodiment, the guidewire's distal end is adjacent to the marking “00” in the fully retracted position. When the proximal end is extended, the distal end will move towards the marking associated with the length extended. In the preferred embodiment, the increments are placed at 1 cm.

The thumb roller may be used by the physician to advance the guidewire from the housing. The outside surface of the housing has a transparent portion through which the physician can visually inspect the length of wire remaining, and in particular, the marking at which the distal end is located.

In one embodiment, the housing has an access port through which a sanitizer may be injected.

In another embodiment of the present invention, the distal end is of one color and the remainder of the guidewire is of a different color.

In another embodiment of the present invention, the housing has a transparent cover.

In another embodiment of the present invention, the guidewire is of one color and an inside surface of the housing is of a different color.

In another embodiment, the housing has a transparent back.

In another embodiment, the housing has a contoured gripping portion.

### BRIEF DESCRIPTION OF THE DRAWINGS

FIG. 1 is top side plan view of the catheter guidewire advancement device of the present invention in accordance with a preferred embodiment with the cover in place.

FIG. 2 is top side plan view of the catheter guidewire advancement device of the present invention in accordance with a preferred embodiment without the cover in place.

FIG. 3 is top side plan view of the catheter guidewire advancement device of the present invention, in accordance with an alternative embodiment, with the cover in place.

FIG. 4 is top side plan view of the catheter guidewire advancement device of the present invention, in accordance with an alternative embodiment, without the cover in place.

FIG. 5 is top side plan view of the catheter guidewire advancement device of the present invention in accordance with another embodiment.

### DESCRIPTION OF THE PREFERRED EMBODIMENT

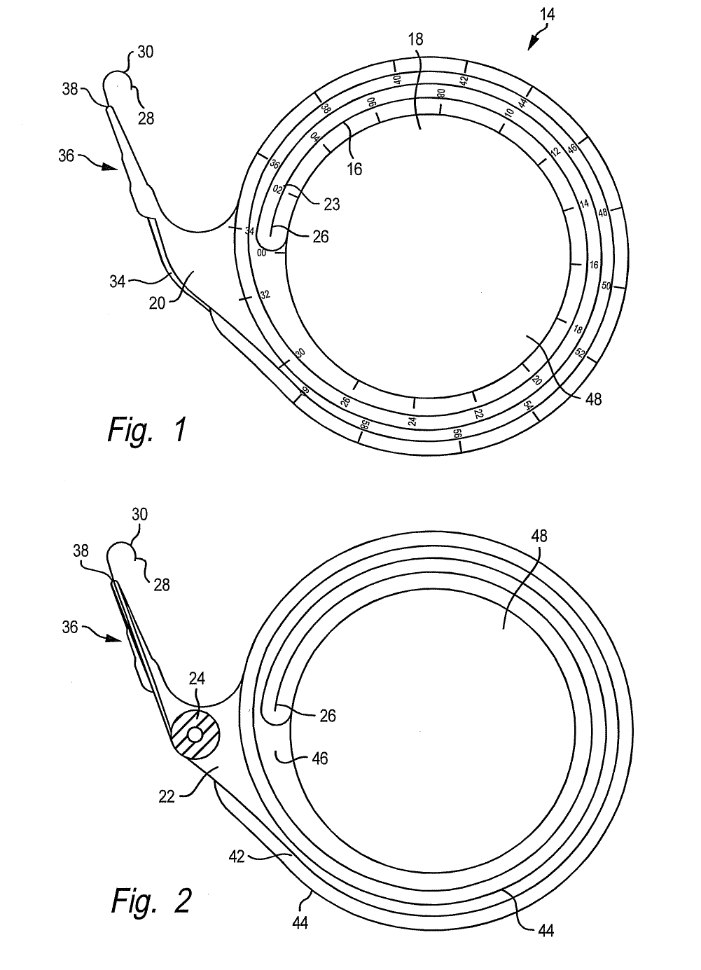

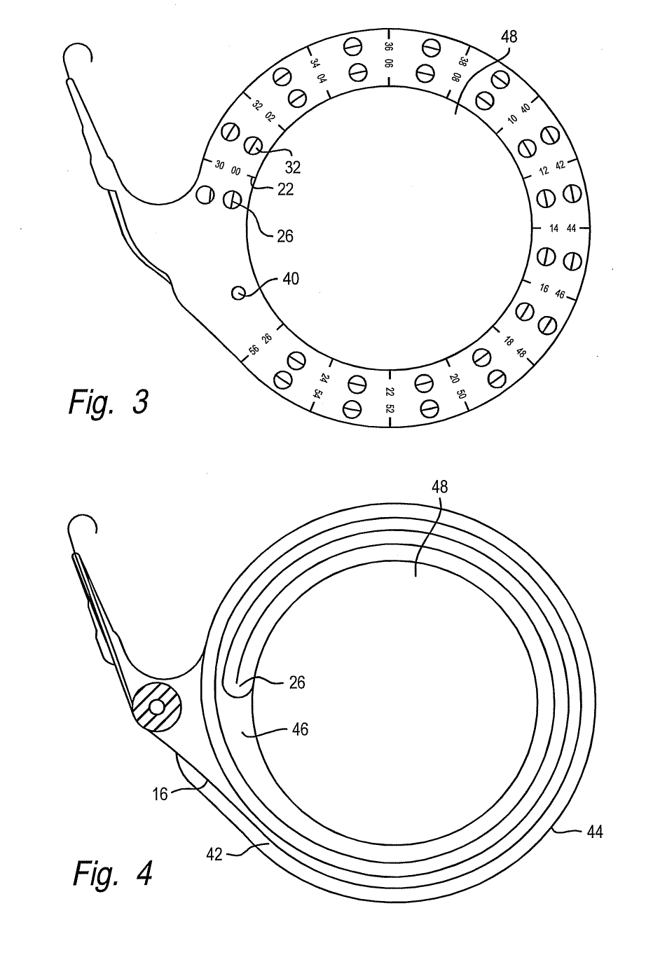

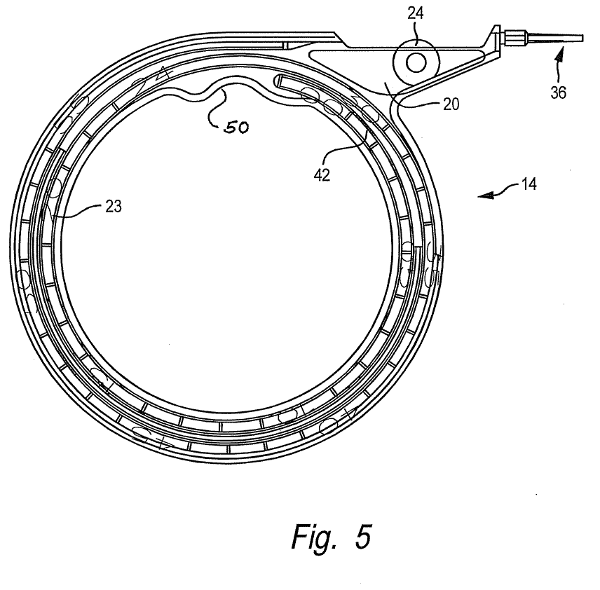

In FIGS. 1 and 2, there is shown the catheter guidewire advancement device 14 in accordance with a preferred embodiment. In FIGS. 3 and 4, an alternative embodiment of the catheter guidewire advancement device 14 is shown. In FIG. 5, still another embodiment is shown. The catheter guidewire advancement device 14 of the present invention is comprised of a partially transparent housing 18, a spiral passage 42 for a guidewire 16, and thumb roller 24. In the preferred embodiment, the housing 18 is generally ring shaped and has a hollow central portion 48 and referring to FIG. 5, in one embodiment, the housing 18 is comprised of a contoured gripping portion 50 which allows the user to easily grasp and maneuver the device 14. In the preferred embodiment, the housing 18 is further comprised of a lower housing 22 coupled to an upper housing 20. The lower 22 and upper 20 housing 18 are each further comprised of a thumb roller opening 34 and a tapered portion 36. The lower housing 22 of the preferred embodiment is comprised of a channel 42 formed by a generally spirally arranged channel side wall 44, a base 46, and a top 20. Because the channel side wall 44 is spirally arranged, the channel side wall 44 forms both sides 44, 44 of the channel 42, while the base 46 and upper housing 20 form a bottom wall 46 and top wall 20, respectively. Thus, the channel is comprised of four walls—the channel side walls 44, 44, the base 46, and upper housing 20. These walls 44, 44, 46, 20 confine the guidewire 16 within the channel 42 such that, when so configured, the guidewire 16 may only be removed though an opening 38 in the tapered portion 36. The channel 42 is structured to coil a guidewire 16 comprised of a proximal end 28 and a distal end 26 within the channel 42. The thumb roller 24 is rotatably positioned distal to the tapered portion 36 between the lower 22 and upper 20 housing 18. The guidewire 16 is coiled within the channel 42 of the housing 18 such that the proximal end 28 extends from the opening 38 in the tapered portion 36. A portion of the guidewire 16 is frictionally coupled to the thumb roller 24 such that when the roller 24 is turned the guidewire 16 moves.

The upper housing 20 has markings 23 calibrated to the length of the guidewire 16 extended from the housing 18. For example, in FIG. 1, the guidewire's 16 distal end 26 is adjacent to the marking 23 “00” which indicates the guidewire 16 is in the fully retracted position. When the proximal end 28 is extended, the distal end 26 will move towards the marking 23 associated with the length extended. By way of example, if the proximal end 28 of the guidewire 16 were extended 4 cm, the distal end 26 would align with the “04” marking 23.

Referring to the figures, the thumb roller 24 may be used by the physician to advance the guidewire 16 from the housing 18. Further, as shown in FIG. 3, the upper surface 20 of the housing 18 has a transparent portion 32 through which the physician can visually inspect the length of guidewire 16 remaining, and in particular, the marking 22 at which the distal end 26 is located.

To facilitate atraumatic passage into a vessel, the proximal end 28 of the guidewire 16 of the preferred embodiment has a “J” tip 30. “J” tip 30 guidewires 16 are preferred by some practitioners over a straight tip because such practitioners find that in certain procedures they are easier to negotiate around anatomical angulations, and, thus, reduce the occurrence of traumatic vessel injury. Although the guidewire 16 of the preferred embodiment has a “J” tip 30, the tip need not be “J” shaped. Rather, the proximal end 28 of the guidewire 16 may be either J-shaped, straight, or some other configuration.

Referring to FIG. 3, in one embodiment, the housing 18 has an access port 40 through which a sanitizer may be injected. In this embodiment, the access port 40 is a small orifice 40 that acts as a one way valve such that the sanitizer may be injected into the housing 18. Injected sanitizer circulates throughout the interior of the housing 18, cleansing and sanitizing the guidewire 16 and housing 18 interior. Injected sanitizer, after circulating throughout the housing 18 interior, exits through the openings 34, 38.

The practitioner determines the length of extended guidewire 16 by comparing the position of the distal end 26 with the markings 23. In another embodiment of the present invention, the distal end 26 is of one color and other portions of the guidewire 16 are of one or more different colors. The contrasting colors may, in some circumstances, allow the physician to more easily see the position of the distal end 26, and, thus, more readily determine the length of guidewire 16 extended.

In another embodiment of the present invention, the guidewire 16 is of one color and an inside surface of the housing 18 is of a different color.

Although in an alternative embodiment (FIG. 3), the physician views the guidewire 16 contained within the housing 18 through openings 32, the device 14 need not have such openings 32. For example, in another embodiment of the present invention, the upper housing 20 is transparent such that the entire length of guidewire 16 may be examined instead of just the portions visible through the openings 32.

In some circumstances, the lower housing 22 may be more convenient for the physician to view, for example, when the physician is left handed. In another embodiment, the lower housing 22 is transparent. In this embodiment, the physician may examine the guidewire 16 through the lower housing 22.

The housing 18 and thumb roller 24 may be formed of any conventional material such as metal or plastic. In the preferred embodiment, the housing 18 and thumb roller 24 are formed from polycarbonate. The guidewire 16 may be any conventional and commercially available guidewire 16 suitable for the desired procedure and capable of being placed within the channel 42 of the housing 18.

The device 14 may be manufactured and assembled for use with many different lengths of guidewire 16. In the preferred embodiment, and as depicted in FIG. 1, the device 14 is assembled for use with a 60 cm guidewire 16. However, the device 14 can be manufactured to accommodate various lengths of guidewire 16.

The operation and use of the guidewire advancement device 14 will now be described. The guidewire advancement device 14 is assembled for use by placing the guidewire 16 within the housing 18 by inserting the guidewire 16 distal end 26 into the end opening 38. The guidewire 16 distal end 26 is then moved over the thumb roller 24 and into the channel 42 until the distal end 26 aligns with the “00” calibration marking 23. The ringed configuration of the device 14 permits the physician to grasp the device 14 through the hollow central portion 48 so that the physician's thumb is positioned above the thumb roller 24. The physician may then use the thumb roller 24 to advance the guidewire 16 from the housing 18 and through a cannula within a blood vessel to a predetermined point within the vascular system by comparing the position of the guidewire 16 distal end 26 with the calibration marking 23 that corresponds with the length of guidewire 16 extended from the housing 18. Once the guidewire 16 has been used, it may be retracted within the housing 18 in a reverse fashion as that which has been described above.

The guidewire advancement device 14 may then be sanitized by injecting a sanitizer through the injection port 40 and circulating the sanitizer throughout the interior of the housing 18, so as to cleanse and sanitize the guidewire 16 and housing 18 interior. The injected sanitizer, after circulating throughout the housing 18 interior, will exit through the openings 34, 38.

While there has been illustrated and described what is, at present, considered to be a preferred embodiment of the present invention, it will be understood by those skilled in the art that various changes and modifications may be made, and equivalents may be substituted for elements thereof without departing from the true scope of the invention. Therefore, it is intended that this invention not be limited to the particular embodiment disclosed as the best mode contemplated for carrying out the invention, but that the invention will include all embodiments falling within the scope of the appended claims.

I claim:

1. A catheter guidewire advancement device comprising:
an at least partially transparent housing comprised of a spirally arranged channel capable of receiving a guidewire, wherein said channel is comprised of first, second, third, and fourth walls, such that said guidewire may be coiled within said channel, said guidewire having a distal end and a proximal end;
wherein said housing is comprised of scale markings, such that said guidewire may be coiled within said channel, said guidewire having a distal end, and a proximal end, such that the distal end, when placed within said channel, aligns with a marking that correlates with a distance between the proximal end of said guidewire and an entrance to said housing; and
a thumb roller.

2. The catheter guidewire advancement device of claim 1 wherein said device has a ringed configuration.

3. The catheter guidewire advancement device of claim 1 further comprised of a tapered end through which the guidewire may be moved.

4. The catheter guidewire advancement device of claim 1 wherein said housing is further comprised of an upper housing and a lower housing and wherein said markings are located on the upper housing.

5. The catheter guidewire advancement device of claim 1 wherein said housing is further comprised of an upper housing and a lower housing and wherein said markings are located on the lower housing.

6. The catheter guidewire advancement device of claim 1 wherein said housing is further comprised of a port through which a liquid may be injected.

7. The catheter guidewire advancement device of claim 1 wherein said housing has a plurality of transparent windows.

8. The catheter guidewire advancement device of claim 1 wherein said distal end is of a first color and a remaining portion of said guidewire is of a second color.

9. The catheter guidewire advancement device of claim 1 wherein said guidewire is of a first color and an inside surface of said housing is of a second color.

10. The catheter guidewire advancement device of claim 2 wherein said housing is further comprised of a contoured gripping portion.

11. A catheter guidewire advancement device comprising:
an at least partially transparent housing comprised of a spirally arranged channel, said channel being comprised of first, second, third, and fourth walls;
a guidewire coiled within said channel, said guidewire having a distal end, and a proximal end;
wherein said housing is comprised of scale markings calibrated such that the distal end of said guidewire, aligns with a marking that correlates with a distance between the proximal end of the guidewire and a predetermined position on said housing; and
a thumb roller.

12. The catheter guidewire advancement device of claim 11 wherein said device has a ringed configuration.

13. The catheter guidewire advancement device of claim 11 further comprised of a tapered end.

14. The catheter guidewire advancement device of claim 11 wherein said housing is further comprised of an upper housing and a lower housing and wherein said markings are located on the upper housing.

15. The catheter guidewire advancement device of claim 11 wherein said housing is further comprised of an upper housing and a lower housing and wherein said markings are located on the lower housing.

16. The catheter guidewire advancement device of claim 11 further comprised of a port through which a liquid may be injected.

17. The catheter guidewire advancement device of claim 11 wherein said housing has a plurality of transparent windows.

18. The catheter guidewire advancement device of claim 11 wherein said distal end is of a first color and a remaining portion of said guidewire is of a second color.

19. The catheter guidewire advancement device of claim 11 wherein said guidewire is of a first color and an inside surface of said housing is of a second color.

20. The catheter guidewire advancement device of claim 12 wherein said housing is further comprised of a contoured gripping portion.
